(19) United States
(12) Patent Application Publication
(10) Pub. No.: US 2026/0083309 A1
(43) Pub. Date: Mar. 26, 2026

(54) LIGHT SOURCE DEVICE AND MEDICAL OBSERVATION SYSTEM

(71) Applicant: SONY GROUP CORPORATION, Tokyo (JP)

(72) Inventors: Tomoyuki OKI, Tokyo (JP); Takuya TSUTSUMI, Tokyo (JP)

(21) Appl. No.: 19/109,574

(22) PCT Filed: Sep. 7, 2023
(86) PCT No.: PCT/JP2023/032698

§ 371 Date: Mar. 7, 2025

(30) Foreign Application Priority Data
Sep. 16, 2022 (JP) 2022-148078

(51) Int. Cl. A61B 1/12; A61B 1/04; A61B 1/06; A61B 90/20

(52) U.S. Cl. CPC A61B 1/128; A61B 1/043; A61B 1/0638; A61B 1/0676; A61B 90/20

(57) ABSTRACT

A light source device according to an aspect of the present disclosure includes a support, a substrate provided on the support, a temperature detecting section that is provided on a surface on a side of the support of the substrate and detects temperature, a light emitting section that is provided on a surface of the support, the surface being opposite to a side of the substrate, and that emits light, and a heat conduction member provided between the temperature detecting section and the support in such a manner as to be in contact with the temperature detecting section and the support.

### FIELD

The present disclosure relates to a light source device and a medical observation system.

### BACKGROUND

Medical observation systems such as an endoscope system and a microscopic surgery system are widely used as systems that observe an internal structure, a surgical site, and the like of an object. These medical observation systems have rapidly spread with development of surgical techniques, and are now indispensable in many medical fields. For example, an endoscope is equipped with a white light source such as a lamp or a light emitting diode (LED) as a light source to illuminate an affected part regardless of whether being a flexible endoscope or a rigid endoscope. In recent years, a function of realizing fluorescence observation of an agent has been added to an endoscope, and the endoscope has evolved from a device that observes an affected part to a device that supports surgery by a doctor (see, for example, Patent Literature 1).

The fluorescence observation of an agent indicates observation of fluorescence generated in response to certain light (excitation light), and some agents are already covered by insurance and widely used. Each agent has a unique absorption spectrum, and fluorescence can be emitted most efficiently when excitation is performed with light having the same wavelength as a peak wavelength of the absorption spectrum. Thus, as a light source device, how to generate excitation light with stable output and wavelength is important. As a light emitting element that is pilot light, a semiconductor laser having a narrow wavelength width and capable of exciting an agent at a pinpoint is suitable.

In general, since a wavelength of a semiconductor laser has a temperature characteristic, a holder capable of temperature control and functioning as a support that holds the semiconductor laser is required to control a wavelength shift. As temperature control, a technology of embedding a lead thermistor in a holder with an ultraviolet adhesive (UV adhesive) or the like, monitoring a holder temperature, and controlling the temperature by a Peltier element or a water cooling system has been widely used.

### CITATION LIST

### Patent Literature

Patent Literature 1: WO 2016/157733 A

### SUMMARY

### Technical Problem

However, in the above-described technology, since the lead thermistor is embedded in the holder with the UV adhesive or the like, it is difficult to embed the lead thermistor in the holder with good reproducibility. For example, it is necessary to remove air in the adhesive to be embedded. When the air remains in a process of embedding the lead thermistor, a reaction rate (thermal reactivity) with heat becomes insufficient depending on an installation state of the lead thermistor, and stable temperature control becomes difficult. In addition, it is very difficult to determine whether all the air has been removed in the embedding process, and there is a large problem in mass productivity.

Thus, the present disclosure provides a light source device and a medical observation system that can realize stable temperature control and improvement in mass productivity.

### Solution to Problem

A light source device according to an aspect of the present disclosure includes a support; a substrate provided on the support; a temperature detecting section that is provided on a surface on a side of the support of the substrate and detects temperature; a light emitting section that is provided on a surface of the support, the surface being on a side opposite to the substrate, and that emits light; and a heat conduction member provided between the temperature detecting section and the support in such a manner as to be in contact with the temperature detecting section and the support.

A medical observation system according to an aspect of the present disclosure includes an imaging device that images an imaging target; and a light source device that generates light emitted to the imaging target, wherein the light source device includes a support, a substrate provided on the support, a temperature detecting section that is provided on a surface on a side of the support of the substrate and that detects temperature, a light emitting section that is provided on a surface of the support, the surface being opposite to a side of the substrate, and that emits light, and a heat conduction member provided between the temperature detecting section and the support in such a manner as to be in contact with the temperature detecting section and the support.

### BRIEF DESCRIPTION OF DRAWINGS

FIG. 1 is a diagram illustrating a configuration example of a medical observation system according to a first embodiment.

FIG. 2 is a front view illustrating a configuration example of an excitation light source according to the first embodiment.

FIG. 3 is a left side view illustrating the configuration example of the excitation light source according to the first embodiment.

FIG. 4 is a right side view illustrating the configuration example of the excitation light source according to the first embodiment.

FIG. 5 is a rear view illustrating the configuration example of the excitation light source according to the first embodiment.

FIG. 6 is a diagram for describing an example of a heat conduction path of the excitation light source according to the first embodiment.

FIG. 7 is a diagram for describing an example of a heat conduction path of an excitation light source of a comparative example according to the first embodiment.

FIG. 8 is a plan view illustrating a configuration example of an excitation light source according to a second embodiment.

FIG. 9 is a front view illustrating a configuration example of an excitation light source according to a third embodiment.

FIG. 10 is a left side view illustrating the configuration example of the excitation light source according to the third embodiment.

FIG. 11 is a left side view illustrating a configuration example of an excitation light source according to a fourth embodiment.

FIG. 12 is a diagram illustrating a substrate and a first heat insulating member according to the fourth embodiment.

FIG. 13 is a diagram for describing thermal collision in an excitation light source according to a fifth embodiment.

FIG. 14 is a front view illustrating a configuration example of the excitation light source according to the fifth embodiment.

FIG. 15 is a plan view illustrating the configuration example of the excitation light source according to the fifth embodiment.

FIG. 16 is a front view illustrating a configuration example of an excitation light source according to a sixth embodiment.

FIG. 17 is a plan view illustrating the configuration example of the excitation light source according to the sixth embodiment.

FIG. 18 is a front view illustrating a configuration example of an excitation light source according to a seventh embodiment.

FIG. 19 is a diagram illustrating an example of a schematic configuration of an endoscope system.

FIG. 20 is a block diagram illustrating an example of a functional configuration of a camera and a camera control unit (CCU) illustrated in FIG. 19.

FIG. 21 is a diagram illustrating an example of a schematic configuration of a microscopic surgery system.

### DESCRIPTION OF EMBODIMENTS

In the following, embodiments of the present disclosure will be described in detail on the basis of the drawings. Note that a system, device, method, and the like according to the present disclosure are not limited by the embodiments. Also, in each of the following embodiments, overlapped description is omitted by assignment of the same reference sign to parts that are basically the same.

Each of one or a plurality of embodiments (including examples and modification examples) described in the following can be performed independently. On the other hand, at least a part of the plurality of embodiments described in the following may be appropriately combined with at least a part of the other embodiments. The plurality of embodiments may include novel features different from each other. Thus, the plurality of embodiments can contribute to solving objects or problems different from each other, and can exhibit effects different from each other.

The present disclosure will be described in the following order of items.
1. First embodiment
1-1. Configuration example of a medical observation system
1-2. Configuration example of an excitation light source
1-3. Example of a heat conduction path of the excitation light source
2. Second embodiment
2-1. Configuration example of an excitation light source
3. Third embodiment
3-1. Configuration example of an excitation light source
4. Fourth embodiment
4-1. Configuration example of an excitation light source
5. Fifth embodiment
5-1. Configuration example of an excitation light source
6. Sixth embodiment
6-1. Configuration example of an excitation light source
7. Seventh embodiment
7-1. Configuration example of an excitation light source
8. Action and effect according to each embodiment
9. Other embodiments
10. Application example
11. Supplementary note

### 1. First Embodiment

### <1-1. Configuration Example of a Medical Observation System>

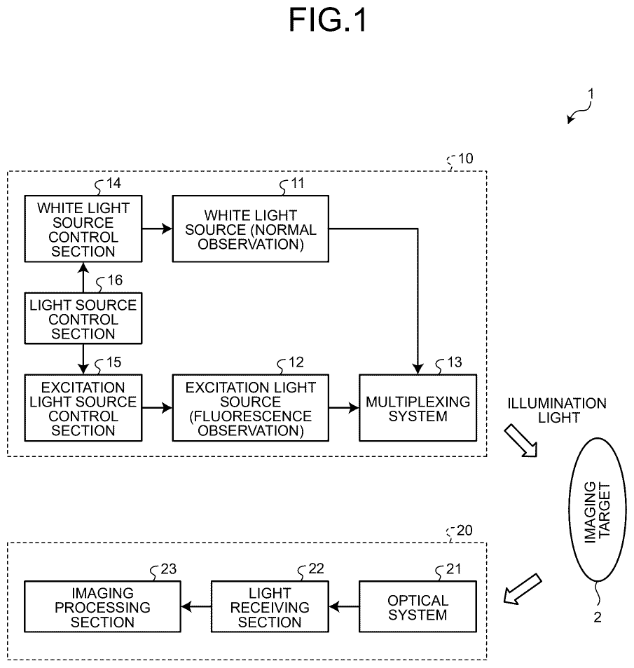

A configuration example of a medical observation system 1 according to the present embodiment will be described with reference to FIG. 1. FIG. 1 is a diagram illustrating the configuration example of the medical observation system 1 according to the present embodiment. Note that examples of the medical observation system 1 include systems such as an endoscope system and a microscope system.

As illustrated in FIG. 1, the medical observation system 1 according to the present embodiment includes an illumination device 10 and an imaging device 20. The medical observation system 1 functions as an image acquisition system that acquires an image of an imaging target 2 to be observed.

### (Illumination Device)

The illumination device 10 includes a white light source 11, an excitation light source (chemical excitation light source) 12, a multiplexing system 13, a white light source control section 14, an excitation light source control section 15, and a light source control section 16.

The white light source 11 is a light source device that emits white light. The white light is usually used for observation. The white light source 11 includes, for example, a lamp, an LED, or the like. Note that a xenon lamp, a halogen lamp, or the like can be used as the lamp, for example.

The excitation light source 12 is a light source device that emits excitation light for chemical excitation. The excitation light is used for fluorescence observation. The excitation light source 12 includes, for example, a semiconductor laser, an LED, or the like. Note that a semiconductor laser having a narrow wavelength width and capable of exciting an agent at a pinpoint is suitable as the excitation light source 12, for example.

The multiplexing system 13 multiplexes the white light emitted from the white light source 11 and the excitation light emitted from the excitation light source 12, and generates illumination light. The illumination light is emitted from the illumination device 10 to the imaging target 2 to be observed.

The white light source control section 14 drives and controls the white light source 11. For example, the white light source control section 14 controls a driving current of the white light source 11 in such a manner as to maintain light quantity of the white light source 11 at a desired value (within a desired range). The white light source control section 14 includes, for example, a central processing unit (CPU), a storage element, and the like.

The excitation light source control section 15 drives and controls the excitation light source 12. For example, the excitation light source control section 15 controls a driving current of the excitation light source 12 in such a manner as to maintain light quantity of the excitation light source 12 at a desired value (within a desired range). The excitation light source control section 15 includes, for example, a CPU, a storage element, and the like.

The light source control section 16 controls the white light source control section 14 and the excitation light source control section 15. For example, the light source control section 16 outputs various control signals to the white light source control section 14 and the excitation light source control section 15. The light source control section 16 includes, for example, a CPU, a storage element, and the like.

### (Imaging Device)

The imaging device 20 includes an optical system 21, a light receiving section 22, and an imaging processing section 23.

The optical system 21 takes in illumination light emitted from the illumination device 10, specifically, the illumination light emitted to and reflected by a target site of the imaging target 2. For example, in a case where the medical observation system 1 is an endoscope system, the optical system 21 is configured to be able to take in the illumination light through an observation window provided at a distal end of an endoscope probe.

The light receiving section 22 is disposed at an image forming position of the optical system 21, receives the illumination light emitted to and reflected by the target site of the imaging target 2, and captures a subject image acquired by imaging of the target site. The light receiving section 22 photoelectrically converts the captured subject image and generates an imaging signal, and outputs the generated imaging signal to the imaging processing section 23. The light receiving section 22 includes, for example, a solid-state image sensor such as a charge coupled device (CCD) or a complementary metal oxide semiconductor (CMOS).

The imaging processing section 23 generates an image on the basis of the imaging signal output from the light receiving section 22, and causes a monitor or the like to display the image, for example. The imaging processing section 23 includes, for example, a CPU, a storage element, and the like.

Note that control sections such as the above-described white light source control section 14, excitation light source control section 15, and light source control section 16, and processing sections such as the imaging processing section 23 may be realized by a processor such as a micro processing unit (MPU) in addition to the CPU, for example. For example, the control sections and the processing sections execute various programs by using a random access memory (RAM) or the like as a work area, but may be realized by an integrated circuit such as a field programmable gate array (FPGA) or an application specific integrated circuit (ASIC). Each of the CPU, MPU, ASIC, and FPGA can be regarded as a processor. Furthermore, the imaging processing section 23 may be realized by a graphics processing unit (GPU) in addition to or instead of the CPU.

Furthermore, the control sections and the processing sections may be realized by specific software instead of specific hardware.

### <1-2. Configuration Example of an Excitation Light Source>

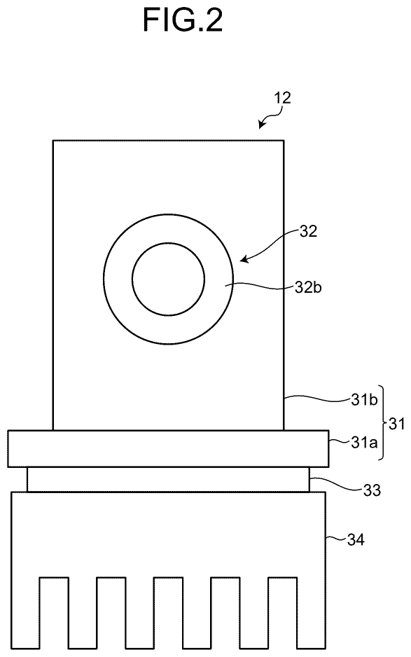

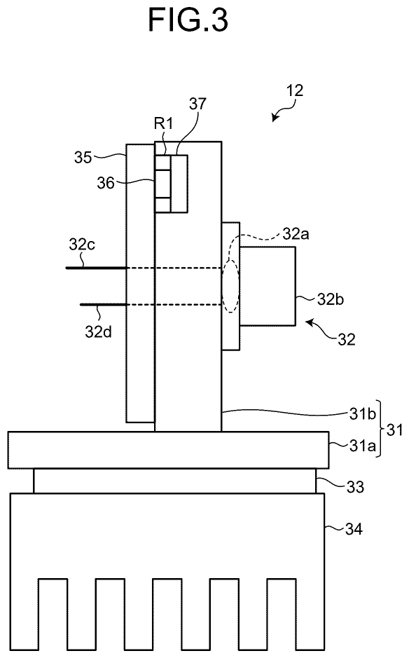

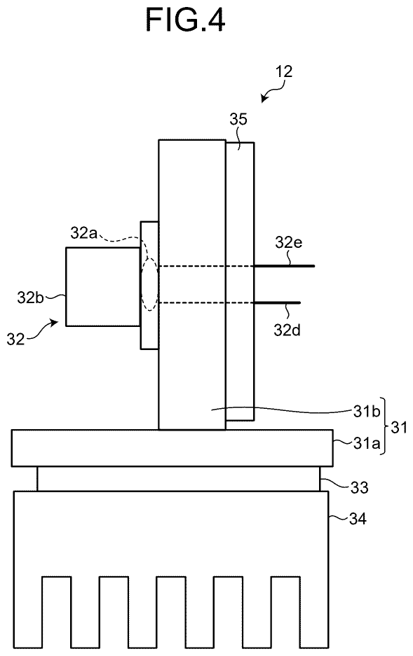

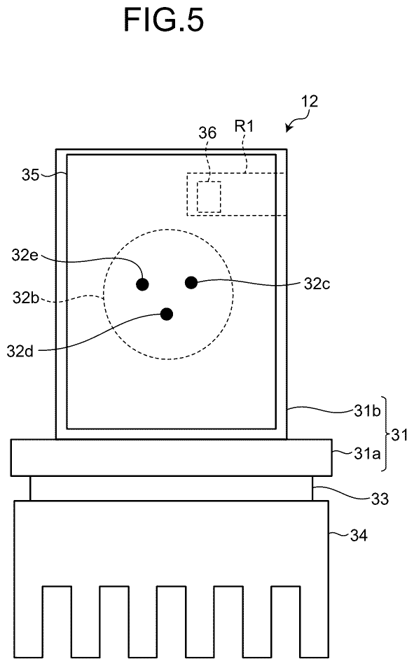

A configuration example of the excitation light source 12 according to the present embodiment will be described with reference to FIG. 2 to FIG. 5. FIG. 2 to FIG. 5 are diagrams illustrating the configuration example of the excitation light source 12 according to the present embodiment. FIG. 2 is a front view, FIG. 3 is a left side view, FIG. 4 is a right side view, and FIG. 5 is a rear view.

As illustrated in FIG. 2 to FIG. 5, the excitation light source 12 according to the present embodiment includes a support 31, a light emitting section 32, a temperature control section 33, a heat dissipation section 34, a substrate 35, a temperature detecting section 36, and a heat conduction member 37.

The support 31 includes a support plate 31a and a support wall 31b. Each of the support plate 31a and the support wall 31b is formed in a rectangular plate shape, for example. For example, the support plate 31a is provided in a horizontal state, and the support wall 31b is provided in a state of standing vertically on an upper surface of the support plate 31a. The support 31 supports and holds each of the sections such as the light emitting section 32 and the substrate 35.

The light emitting section 32 includes a light emitting element 32a, a cover part 32b, and a plurality of terminals 32c, 32d, and 32e. The light emitting element 32a is provided in front of the support wall 31b of the support 31. The light emitting element 32a is a light emitting element that emits excitation light capable of exciting a phosphor. The light emitting element 32a is realized by, for example, a semiconductor laser, an LED, or the like. The cover part 32b is provided in front of the support wall 31b in such a manner as to cover the light emitting element 32a. Note that the cover part 32b has, on a front side, a glass part through which the light emitted from the light emitting element 32a passes. Each of the terminals 32c, 32d, and 32e is, for example, a lead that extends from the light emitting element 32a and is to supply power to the light emitting element 32a. These terminals 32c, 32d, and 32e are electrically connected to the substrate 35 (such as a printed circuit board).

The temperature control section 33 is a temperature control section that controls a temperature of the support 31. The temperature control section 33 is provided on a lower surface of the support plate 31a of the support 31 in such a manner as to be in contact with the lower surface. The temperature control section 33 includes, for example, a Peltier element, an air cooling device, a water cooling device, or the like. Note that the Peltier element generates heat on one surface of the element and absorbs heat on the opposite surface. The heat generating surface and the heat absorbing surface of the Peltier element are switched when a direction of a DC current is changed. Thus, in a case where the Peltier element is used as the temperature control section 33, the support 31 can be heated or cooled by changing of the direction of the direct current.

The heat dissipation section 34 is a member that dissipates heat. The heat dissipation section 34 is provided in such a manner as to be in contact with the temperature control section 33, and is provided on a lower surface of the temperature control section 33 in such a manner as to be in contact with the lower surface, for example. Thus, the heat dissipation section 34 dissipates heat from the support 31 and the temperature control section 33. The heat dissipation section 34 includes, for example, a heat sink or the like.

The substrate 35 is provided on a back surface of the support wall 31b of the support 31. The substrate 35 includes, for example, a printed circuit board (wiring board). For example, the substrate 35 may be provided with the above-described excitation light source control section 15 that supplies current to the light emitting element 32a of the light emitting section 32. In this case, the excitation light source control section 15 is mounted on and electrically connected to the substrate 35 by soldering.

The temperature detecting section 36 is provided on a front surface of an upper part of the substrate 35 (surface on a side of the support wall 31b of the substrate 35). The temperature detecting section 36 includes, for example, a chip thermistor or the like. The temperature detecting section 36 is mounted on and electrically connected to the substrate 35 by soldering, and transmits a detected temperature to the excitation light source control section 15 on the substrate 35, for example. The excitation light source control section 15 controls the temperature control section 33 on the basis of the received temperature information. For example, the excitation light source control section 15 controls the temperature control section 33 in such a manner as to maintain a temperature of the support 31, that is, the light emitting section 32 at a desired temperature (for example, within a desired temperature range). As an example, the excitation light source control section 15 controls the temperature control section 33 in such a manner as to cool the support 31 by the temperature control section 33 in a case where the received temperature is higher than the desired temperature, and to heat the support 31 by the temperature control section 33 in a case where the received temperature is lower than the desired temperature.

Here, the support wall 31b of the support 31 has a housing chamber R1 that houses the temperature detecting section 36. The housing chamber R1 is a recess part formed in the back surface of the support wall 31b (surface on a side of the substrate 35 of the support wall 31b). This recess part is formed in such a manner as to extend to a left side surface of the support wall 31b. As a result, a left side surface of the housing chamber R1 is opened, and a worker, an inspector, and the like of a manufacturing process can visually confirm the heat conduction member 37 in the housing chamber R1 from a side of the left side surface of the support wall 31b.

The heat conduction member 37 is a member having thermal conductivity. The heat conduction member 37 is provided between the temperature detecting section 36 and the support wall 31b of the support 31 in the housing chamber R1 in such a manner as to be in contact with the temperature detecting section 36 and the support wall 31b. The heat conduction member 37 is made of, for example, a heat conduction sheet, a graphite sheet, or thermal grease.

According to the excitation light source 12 having such a configuration, the temperature detecting section 36 is provided on the substrate 35 and housed in the housing chamber R1, and is not embedded in the support wall 31b with a UV adhesive or the like as in the related art. Furthermore, since the temperature detecting section 36 can receive heat from the support wall 31b via the heat conduction member 37, thermal reactivity (thermal responsiveness) of the temperature detecting section 36 can be improved. Thus, the temperature detecting section 36 can accurately detect temperature. As a result, since it becomes possible to perform temperature control on the basis of the accurate temperature, and it is possible to realize stable temperature control. In addition, since it is not necessary to determine whether all the air is removed in the embedding process as in the related art, it is possible to realize improvement in mass productivity.

In addition, the recess part of the housing chamber R1 is formed in such a manner as to extend to the left side surface of the support wall 31b, and the left side surface of the housing chamber R1 is opened. As a result, since the worker, the inspector, and the like of the manufacturing process can visually confirm the heat conduction member 37 in the housing chamber R1 from the side of the left side surface of the support wall 31b, inspection work of inspecting presence or absence, a state, and the like of the heat conduction member 37 can be simplified.

Note that the configuration related to the excitation light source 12 may be applied to the white light source 11. For example, in a case where an LED is used as the white light source 11, the configuration related to the excitation light source 12 can be applied to the white light source 11.

### <1-3. Example of a Heat Conduction Path of the Excitation Light Source>

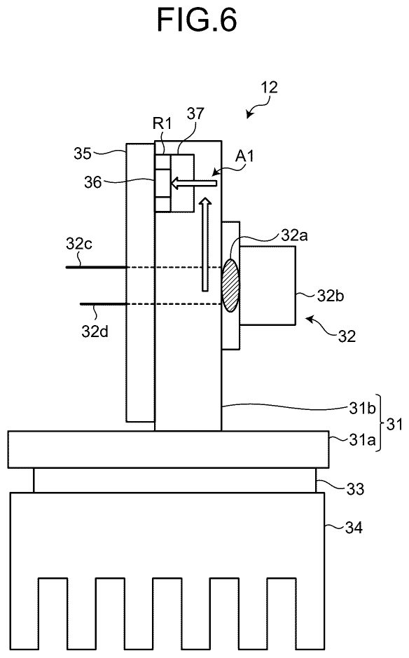

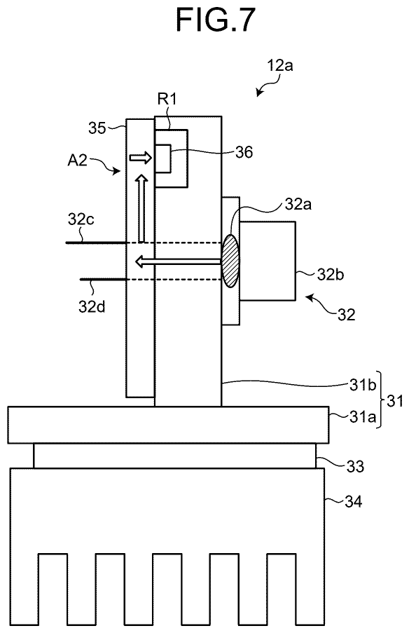

A heat conduction path A1 of the excitation light source 12 according to the present embodiment will be described with reference to FIG. 6 and FIG. 7. FIG. 6 is a diagram illustrating an example of the heat conduction path A1 of the excitation light source 12 according to the present embodiment. FIG. 7 is a diagram illustrating an example of a heat conduction path A2 of an excitation light source 12a of a comparative example according to the present embodiment. The excitation light source 12a of the comparative example includes no heat conduction member 37.

As illustrated in FIG. 6, the heat conduction path A1 of the excitation light source 12 according to the present embodiment is a path through which heat generated in the light emitting element 32a of the light emitting section 32 passes through the support wall 31b and reaches the temperature detecting section 36 via the heat conduction member 37. On the other hand, as illustrated in FIG. 7, the heat conduction path A2 of the excitation light source 12a of the comparative example is a path in which heat generated in a light emitting element 32a of a light emitting section 32 passes through a support wall 31b, enters a substrate 35, passes through the substrate 35, and reaches the temperature detecting section 36.

Since being shorter than the heat conduction path A2 including the substrate 35 and illustrated in FIG. 7, the heat conduction path A1 including the heat conduction member 37 and illustrated in FIG. 6 is a path in which a reaction rate (response speed) of heat of the temperature detecting section 36 is faster than that of the heat conduction path A2. That is, when the heat conduction member 37 is provided between the temperature detecting section 36 and the support wall 31b, the temperature detecting section 36 on the substrate 35 receives heat from the support wall 31b via the heat conduction member 37 without the substrate 35. Thus, the reaction rate of heat is higher than that of a case where the heat passes through the substrate 35. As a result, the temperature detecting section 36 can accurately detect the temperature, that is, the temperature of the light emitting section 32 (such as the light emitting element 32a).

### 2. Second Embodiment

### <2-1. Configuration Example of an Excitation Light Source>

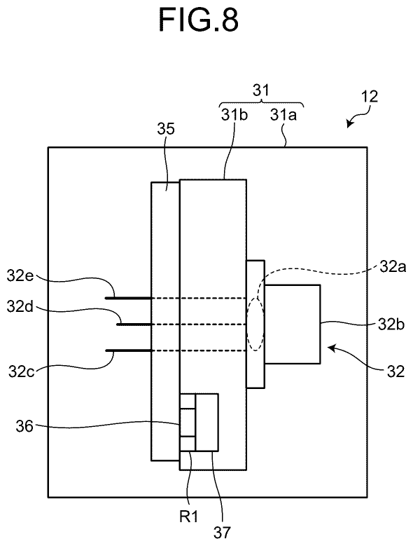

A configuration example of an excitation light source 12 according to the present embodiment will be described with reference to FIG. 8. FIG. 8 is a plan view illustrating the configuration example of the excitation light source 12 according to the present embodiment. Note that parts different from those of the first embodiment will be basically described in the present embodiment.

As illustrated in FIG. 8, in the excitation light source 12 according to the present embodiment, a housing chamber R1 is a recess part formed in a back surface of a support wall 31b (surface on a side of a substrate 35 of a support wall 31b). This recess part is formed in such a manner as to extend to an upper surface of the support wall 31b. As a result, since a worker, an inspector, and the like of a manufacturing process can visually confirm a heat conduction member 37 in the housing chamber R1 from an upper surface side of the support wall 31b, inspection work of inspecting presence or absence, a state, and the like of the heat conduction member 37 can be simplified.

Note that the recess part of the housing chamber R1 may be formed in such a manner as to extend to a right side surface other than the upper surface and a left side surface of the support wall 31b, and only needs to be formed in such a manner as to extend to an exposed surface that is an end surface such as the upper surface, the left side surface, or the right side surface of the support wall 31b. However, in order to simplify the inspection work, it is desirable that the recess part is formed to extend to the upper surface of the support wall 31b. This is because it is easier for the worker, the inspector, and the like to visually recognize the heat conduction member 37 in the housing chamber R1 from the upper surface side than a case where the heat conduction member 37 in the housing chamber R1 is visually recognized from the other surface side.

### 3. Third Embodiment

### <3-1. Configuration Example of an Excitation Light Source>

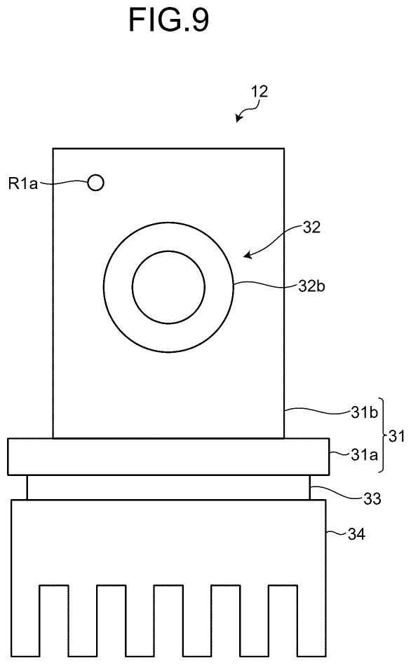

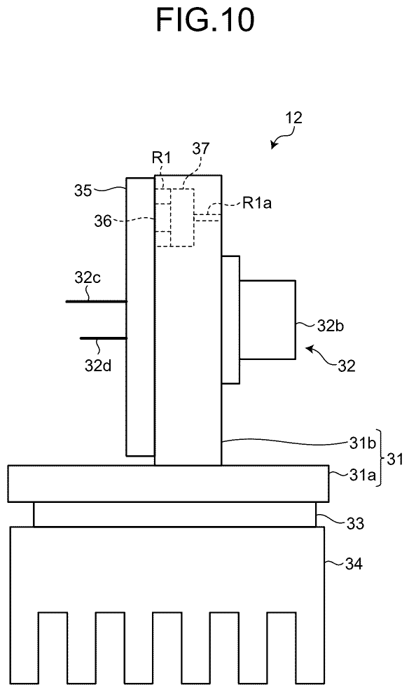

A configuration example of an excitation light source 12 according to the present embodiment will be described with reference to FIG. 9 and FIG. 10. FIG. 9 and FIG. 10 are diagrams illustrating the configuration example of the excitation light source 12 according to the present embodiment. FIG. 9 is a front view, and FIG. 10 is a left side view. Note that parts different from those of the first embodiment will be basically described in the present embodiment.

As illustrated in FIG. 9 and FIG. 10, in the excitation light source 12 according to the present embodiment, a housing chamber R1 is a recess part formed in a back surface of a support wall 31b (surface on a side of a substrate 35 of a support wall 31b). This recess part is not formed in such a manner as to extend to a left side surface of the support wall 31b, and the left side surface of the recess part is in a closed state. On the other hand, the support wall 31b has a through hole R1a extending from a front surface of the support wall 31b (surface on a side of a light emitting section 32 of the support wall 31b) to the housing chamber R1. As a result, since a worker, an inspector, and the like of a manufacturing process can visually confirm a heat conduction member 37 in the housing chamber R1 from the through hole R1a of the support wall 31b, inspection work of inspecting presence or absence, and the like of the heat conduction member 37 can be simplified.

Note that the through hole R1a may be formed in such a manner as to extend from an upper surface, a left side surface, or a right side surface of the support wall 31b other than the front surface of the support wall 31b to the housing chamber R1, and only needs to be formed in such a manner as to extend from an exposed surface that is an end surface such as the front surface, the upper surface, the left side surface, or the right side surface of the support wall 31b to the housing chamber R1. However, in order to simplify the inspection work, it is desirable that the through hole R1a is formed to extend from the upper surface of the support wall 31b to the housing chamber R1. This is because it is easier for the worker, the inspector, and the like to visually recognize the heat conduction member 37 in the housing chamber R1 from the upper surface side than a case where the heat conduction member 37 in the housing chamber R1 is visually recognized from the other surface side. In addition, the through hole R1a may be provided in the housing chamber R1 of the recess part according to the other embodiments, or the through hole R1a according to the present embodiment may be combined with the other embodiments.

### 4. Fourth Embodiment

### <4-1. Configuration Example of an Excitation Light Source>

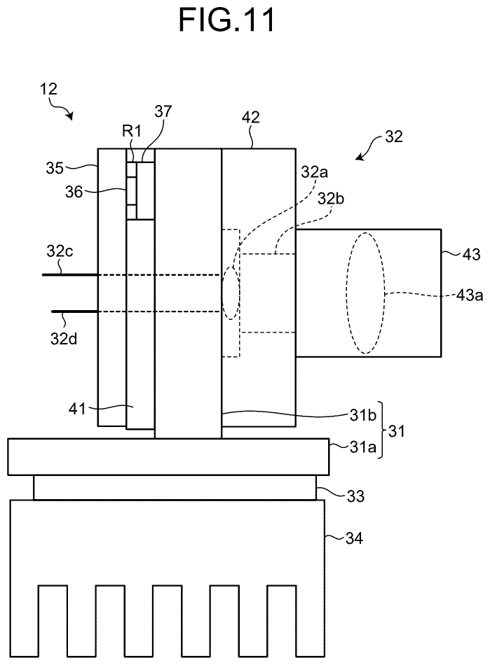

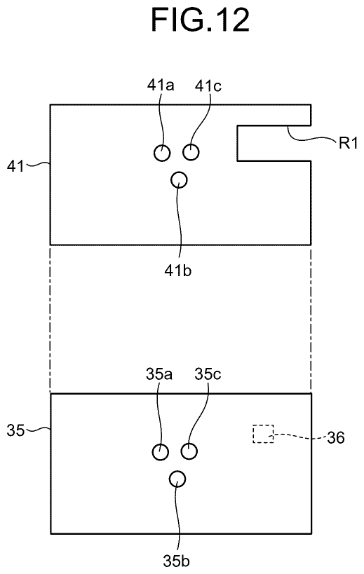

A configuration example of an excitation light source 12 according to the present embodiment will be described with reference to FIG. 11 and FIG. 12. FIG. 11 is a left side view illustrating the configuration example of the excitation light source 12 according to the present embodiment. FIG. 12 is a diagram illustrating a substrate 35 and a first heat insulating member 41 according to the present embodiment. Note that parts different from those of the first embodiment will be basically described in the present embodiment.

As illustrated in FIG. 11, the excitation light source 12 according to the present embodiment includes the first heat insulating member 41, a second heat insulating member 42, and an optical member 43. The optical member 43 includes, for example, a lens barrel or the like. The optical member 43 includes a lens 43a. The optical member 43 is attached to a light emitting section 32, for example.

The first heat insulating member 41 is provided between the substrate 35 and the support wall 31b. For example, the first heat insulating member 41 is in contact with the substrate 35 and the support wall 31b, and is formed in such a manner as to surround a temperature detecting section 36 and a heat conduction member 37. Instead of the support wall 31b according to the first embodiment, the first heat insulating member 41 includes a housing chamber R1. That is, the support wall 31b according to the present embodiment does not have the housing chamber R1. The first heat insulating member 41 is made of, for example, a plastic material.

The second heat insulating member 42 is provided between the support wall 31b and the optical member 43. For example, the second heat insulating member 42 is in contact with the support wall 31b and the optical member 43, and is formed in such a manner as to surround a cover part 32b of the light emitting section 32. The second heat insulating member 42 is made of, for example, a plastic material.

As illustrated in FIG. 12, the housing chamber R1 of the first heat insulating member 41 is a cutout part formed in the first heat insulating member 41. A left side surface of the first heat insulating member 41 is cut out and the cutout part is formed. As a result, since a worker, an inspector, and the like of a manufacturing process can visually confirm the heat conduction member 37 in the housing chamber R1 from a side of the left side surface of the first heat insulating member 41, inspection work of inspecting presence or absence, a state, and the like of the heat conduction member 37 can be simplified.

The first heat insulating member 41 has a plurality of through holes 41a, 41b, and 41c, and the substrate 35 has a plurality of through holes 35a, 35b, and 35c. The through holes 41a, 41b, and 41c and the through holes 35a, 35b, and 35c are respectively formed at positions facing each other. A terminal 32e is inserted into each of the through holes 41a and 35a, a terminal 32d is inserted into each of the through holes 41b and 35b, and a terminal 32c is inserted into each of the through holes 41c and 35c.

Note that the cutout part of the housing chamber R1 may be formed by cutting out of an upper surface or a right side surface other than the left side surface of the first heat insulating member 41, and only needs to be formed by cutting out of an exposed surface that is an end surface such as the left side surface, the upper surface, or the right side surface of the first heat insulating member 41. However, in order to simplify the inspection work, it is desirable that the upper surface of the first heat insulating member 41 is cut out and the cutout part is formed. This is because it is easier for a person such as the worker, the inspector, or the like to visually recognize the heat conduction member 37 in the housing chamber R1 from a side of the upper surface than a case where the heat conduction member 37 in the housing chamber R1 is visually recognized from the other surface side.

Here, there is a case where a temperature of the excitation light source 12 becomes lower than a dew point temperature depending on an environment in which the excitation light source 12 is used, and there is a case where dew condenses on a support 31 of the excitation light source 12. For example, due to dew condensation generated when the support 31 becomes the dew point temperature or lower, the temperature detecting section 36 may not be able to correctly detect the temperature. Thus, by providing the first heat insulating member 41 in such a manner as to surround the temperature detecting section 36 and the heat conduction member 37 between the support wall 31b of the support 31 and the substrate 35, it is possible to control dew condensation on the temperature detecting section 36 and the substrate 35 due to heat conduction of the support 31 and to control erroneous detection of the temperature by the temperature detecting section 36 due to the dew condensation. For example, it is possible to control a risk of an electric short circuit of the temperature detecting section 36, the substrate 35, and the like. In addition, by providing the second heat insulating member 42 between the support wall 31b of the support 31 and the optical member 43 in such a manner as to surround the cover part 32b of the light emitting section 32, dew condensation on the optical member 43 due to the heat conduction of the support 31 can be controlled. For example, fogging or the like of the lens 43a of the optical member 43 can be controlled.

Note that although both the first heat insulating member 41 and the second heat insulating member 42 are provided on the support 31, this is not a limitation. For example, only one of the first heat insulating member 41 or the second heat insulating member 42 may be provided on the support 31 as necessary. For example, only the first heat insulating member 41 may be provided on the support 31 in a case where the optical member 43 is waterproof.

### 5. Fifth Embodiment

### <5-1. Configuration Example of an Excitation Light Source>

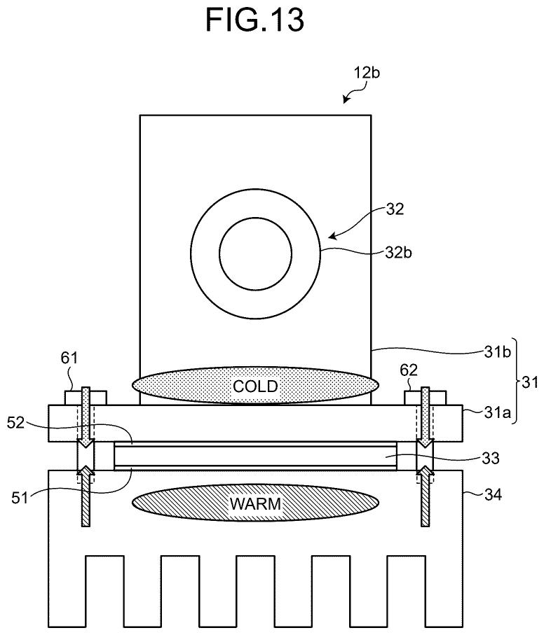

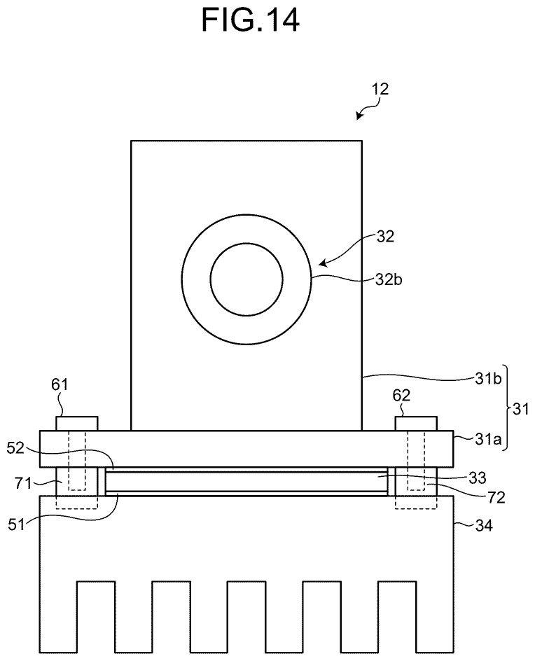

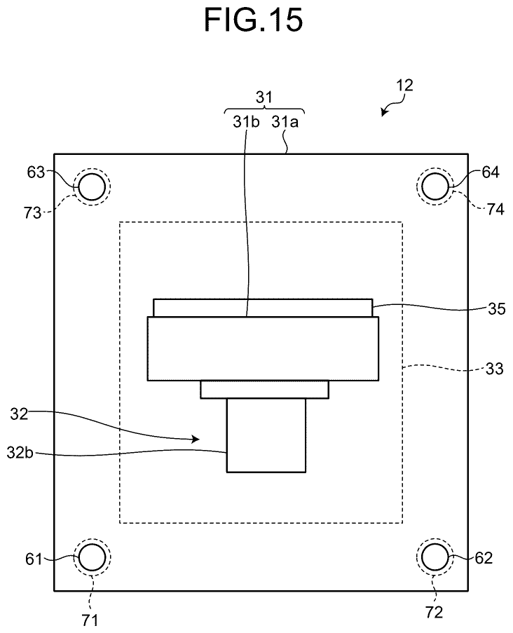

A configuration example of an excitation light source 12 according to the present embodiment will be described with reference to FIG. 13 to FIG. 15. FIG. 13 is a diagram for describing thermal collision in an excitation light source 12b according to the present embodiment. FIG. 14 and FIG. 15 are diagrams illustrating the configuration example of the excitation light source 12 according to the present embodiment. FIG. 14 is a front view, and FIG. 15 is a plan view. Note that parts different from those of the first embodiment will be basically described in the present embodiment.

As illustrated in FIG. 13, a support 31 of the excitation light source 12b is fixed to a heat dissipation section 34 by a plurality of fixing members 61 and 62 via a temperature control section 33. Although only the two fixing members 61 and 62 are illustrated in the example of FIG. 13, actually, the fixing members are respectively provided at four corners of a support plate 31a of the support 31. Each of the fixing members 61 and 62 is formed of a member having thermal conductivity, such as a metal screw. Since these fixing members 61 and 62 serve as heat paths, thermal collision is generated. In the example of FIG. 13, the support 31 is cold (COLD), and the heat dissipation section 34 is warm (WARM). In this case, thermal collision is generated in each of the fixing members 61 and 62. This thermal collision significantly lowers efficiency of the temperature control section 33.

The temperature control section 33 of the excitation light source 12b is provided between the support 31 and the heat dissipation section 34, and grease having thermal conductivity is used at that time. The grease is an example of liquid lubricating oil. Thus, a grease layer 51 is provided between the temperature control section 33 and the heat dissipation section 34, and a grease layer 52 is also provided between the temperature control section 33 and the support 31. The temperature control section 33 has a variation in thickness (height). In the example of FIG. 13, the thickness of the temperature control section 33 is a length of the temperature control section 33 in a vertical direction. In a case where it is desired to fix a height of emission light (such as laser light) of the light emitting section 32, when the temperature control section 33 is fixed between the support 31 and the heat dissipation section 34, it is necessary to make a vertical separation distance between the support 31 and the heat dissipation section 34 constant. However, it is difficult to make the vertical separation distance between the support 31 and the heat dissipation section 34 constant due to the thickness variation of the temperature control section 33.

In order to avoid such thermal collision and thickness variation, as illustrated in FIG. 14 and FIG. 15, heat insulating members 71, 72, 73, and 74 (71 to 74) are provided for fixing members 61, 62, 63, and 64 (61 to 64) of the excitation light source 12, respectively. The heat insulating members 71 to 74 function as control members that control thermal collision in the fixing members 61 to 64, and further function as height determination members that make the vertical separation distance between the support 31 and the heat dissipation section 34 constant. Each of such heat insulating members 71 to 74 will be described in detail. Note that each of the fixing members 61 to 64 is formed of, for example, a metal screw having thermal conductivity.

As illustrated in FIG. 14 and FIG. 15, the heat dissipation section 34 of the excitation light source 12 includes the plurality of heat insulating members 71 to 74. These heat insulating members 71 to 74 are respectively provided and fixed at the four corners of the upper surface of the heat dissipation section 34. In the example of FIG. 14, recess parts are respectively formed at the four corners of the heat dissipation section 34, and the heat insulating members 71 to 74 are embedded and fixed in the recess parts. Each of the heat insulating members 71 to 74 has, for example, a female screw into which each fixing member 61 such as a male screw is inserted. Such heat insulating members 71 to 74 are made of, for example, a plastic material or the like. The heat insulating members 71 to 74 are placed between the support plate 31a and the heat dissipation section 34 and respectively prevent contact between the fixing members 61 to 64 and the heat dissipation section 34.

The support 31 is fixed to the heat dissipation section 34 by the plurality of fixing members 61 to 64 via the temperature control section 33. For example, the fixing members 61 to 64 such as the male screws are respectively inserted into female screws of the heat insulating members 71 to 74 via the through holes (or female screws) of the support plate 31a of the support 31, and the support 31 is fastened to the heat dissipation section 34 via the temperature control section 33. At this time, each of the heat insulating members 71 to 74 makes the vertical separation distance between the support 31 and the heat dissipation section 34 constant. As a result, the height of the emission light (such as laser light) of the light emitting section 32 can be fixed. In addition, since the heat insulating members 71 to 74 respectively control thermal collision in the fixing members 61, 62, 63, and 64, the efficiency of the temperature control section 33 can be improved.

### 6. Sixth Embodiment

### <6-1. Configuration Example of an Excitation Light Source>

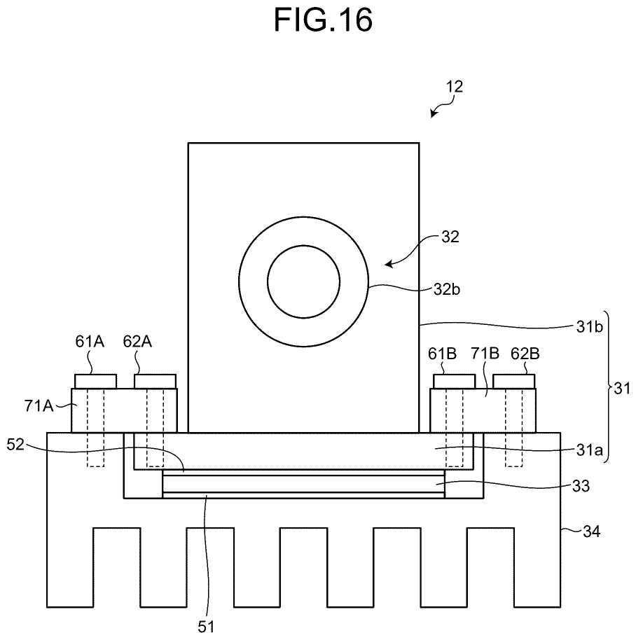

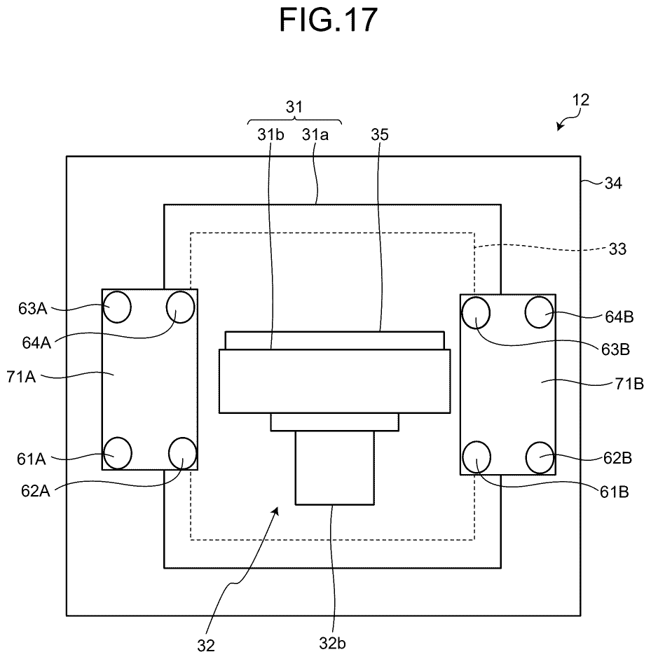

A configuration example of an excitation light source 12 according to the present embodiment will be described with reference to FIG. 16 and FIG. 17. FIG. 16 and FIG. 17 are diagrams illustrating the configuration example of the excitation light source 12 according to the present embodiment. FIG. 16 is a front view, and FIG. 17 is a plan view. Note that parts different from those of the fifth embodiment will be basically described in the present embodiment.

As illustrated in FIG. 16 and FIG. 17, a support 31 of the excitation light source 12 is fixed to a heat dissipation section 34 by a plurality of fixing members 61A to 64A and 61B to 64B and a plurality of heat insulating members 71A and 71B via a temperature control section 33. The heat dissipation section 34 is formed in such a manner that an outer peripheral portion (outer peripheral region) of an upper surface of the heat dissipation section 34 has the same height as an upper surface of a support plate 31a of the support 31. A plane size of the support plate 31a is formed to be narrower than that of the support plate 31a according to the fifth embodiment. As a result, downsizing of the support 31 can be realized. Note that the support plate 31a according to the fifth embodiment needs to be larger than a plane size of the temperature control section 33 in order to fix the support 31 and the heat dissipation section 34 by the respective fixing members 61 to 64 with the temperature control section 33 interposed therebetween.

The heat insulating members 71A and 71B are provided over the upper surfaces of the support plate 31a of the support 31 and the heat dissipation section 34. The heat insulating members 71A and 71B are provided at positions that are respectively on both sides of the support plate 31a and that face each other. Such heat insulating members 71A and 71B are made of, for example, a plastic material or the like. The fixing members 61A to 64A are provided at four corners of the heat insulating member 71A, and the fixing members 61B to 64B are provided at the four corners of the heat insulating member 71A. For example, the fixing members 61A and 63A such as male screws are respectively inserted into female screws formed in the heat dissipation section 34, and the fixing members 62A and 64A such as male screws are respectively inserted into female screws formed in the support plate 31a. Similarly, the fixing members 62B and 64B such as male screws are respectively inserted into female screws formed in the heat dissipation section 34, and the fixing members 61B and 63B such as male screws are respectively inserted into female screws formed in the support plate 31a. The heat insulating members 71A and 71B are provided over the upper surfaces of the support plate 31a and the heat dissipation section 34 and prevent contact between the fixing members 61A to 64A and 61B to 64B.

The support 31 is fixed to the heat dissipation section 34 by the fixing members 61A to 64A and 61B to 64B and the heat insulating members 71A and 71B via the temperature control section 33. For example, the fixing members 61A to 64A and 61B to 64B such as the male screws are inserted into the female screws of the heat dissipation section 34 and the support plate 31a via the through holes (or female screws) of the heat insulating members 71A and 71B, and the support 31 is fastened to the heat dissipation section 34 via the temperature control section 33. At this time, each of the heat insulating members 71A and 71B makes the vertical separation distance between the support 31 and the heat dissipation section 34 constant. As a result, a height of emission light (such as laser light) of a light emitting section 32 can be fixed. In addition, since the heat insulating members 71A and 71B control thermal collision in the fixing members 61A to 64A and 61B to 64B, efficiency of the temperature control section 33 can be improved.

### 7. Seventh Embodiment

### <7-1. Configuration Example of an Excitation Light Source>

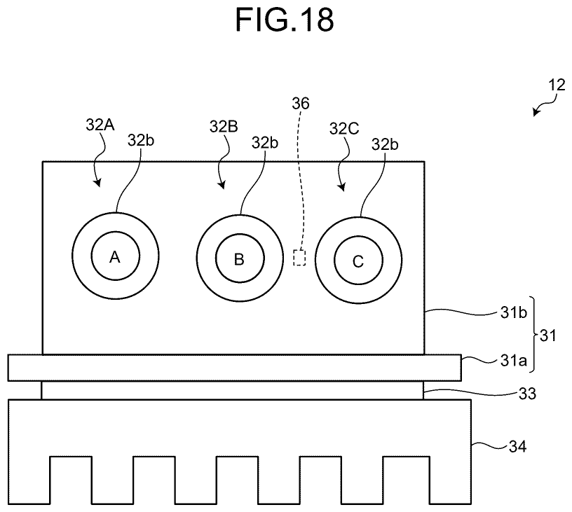

A configuration example of an excitation light source 12 according to the present embodiment will be described with reference to FIG. 18. FIG. 18 is a front view illustrating the configuration example of the excitation light source 12 according to the present embodiment. Note that parts different from those of the first embodiment will be basically described in the present embodiment.

As illustrated in FIG. 18, the excitation light source 12 according to the present embodiment includes three light emitting sections 32A, 32B, and 32C. The light emitting sections 32A, 32B, and 32C are arranged at predetermined intervals in a longitudinal direction and provided on a support wall 31b. In addition, a temperature detecting section 36 is provided at a place where a calorific value is large, for example, between the two light emitting sections 32B and 32C. The calorific values of the light emitting sections 32A, 32B, and 32C increase in order of the light emitting section 32A, the light emitting section 32B, and the light emitting section 32C, for example. Note that as light emitting elements 32a of the light emitting sections 32A, 32B, and 32C, for example, a red semiconductor laser, a green semiconductor laser, and a blue semiconductor laser are used. As described above, by using the various light emitting sections 32A, 32B, and 32C that emit light of different wavelengths, it is possible to generate and emit several types of excitation light respectively corresponding to various phosphors.

A place where temperature is detected is easily limited in the conventional lead thermistor. Compared to the lead thermistor, a degree of freedom in installation of the temperature detecting section 36 provided on the substrate 35 is high, and a place where the temperature is detected can be appropriately changed. Thus, the temperature detecting section 36 can be disposed at a place which is in a vicinity of the light emitting section 32C having the largest calorific value among the plurality of light emitting sections 32A, 32B, and 32C and in which the temperature easily rises in the support 31, for example, between the two light emitting sections 32B and 32C.

As a result, even when the temperature detecting section 36 is not installed for each of the light emitting sections 32A, 32B, and 32C, each of the light emitting sections 32A, 32B, and 32C can be used at the desired temperature (for example, within the desired temperature range) with the one temperature detecting section 36, and a life can be secured.

### 8. Action and Effect According to Each Embodiment

As described above, according to the present embodiment, the excitation light source 12 that is an example of the light source device includes the support 31, the substrate 35 (such as a printed circuit board) provided on the support 31, the temperature detecting section 36 that is provided on the surface on the side of the support 31 of the substrate 35 and that detects temperature, the light emitting section 32 that is provided on the surface on the opposite side of the substrate 35 of the support 31 and that emits light, and the heat conduction member 37 that is provided between the temperature detecting section 36 and the support 31 in such a manner as to be in contact with the temperature detecting section 36 and the support 31. As a result, the temperature detecting section 36 is provided on the substrate 35, and is not embedded in the support wall 31b with the UV adhesive or the like as in the related art. In addition, since the heat generated by the light emitting section 32 is transferred to the temperature detecting section 36 through the heat conduction path A1 including the heat conduction member 37, the reaction rate of the heat of the temperature detecting section 36 is high, and the temperature detecting section 36 can accurately detect the temperature, that is, the temperature of the light emitting section 32. Thus, it is possible to perform temperature control on the basis of the accurate temperature, and stable temperature control can be realized. In addition, since it is not necessary to determine whether all the air is removed in the embedding process as in the related art, it is possible to realize improvement in mass productivity.

Furthermore, the support 31 may have the housing chamber R1 that houses the temperature detecting section 36 (see FIG. 3 and FIG. 5). As a result, even when the temperature detecting section 36 is provided at any position on the surface on the side of the support 31 of the substrate 35, the housing chamber R1 may be formed in accordance with the position, and a degree of freedom in installation of the temperature detecting section 36 can be improved.

In addition, the housing chamber R1 may be a recess part formed in the surface on the side of the substrate 35 of the support 31 (see FIG. 3 and FIG. 5). Thus, the housing chamber R1 can be easily formed.

In addition, the recess part may be formed in such a manner as to extend to the exposed surface of the support 31 (see FIG. 3 and FIG. 5). As a result, since the worker, the inspector, and the like of the manufacturing process can visually confirm the heat conduction member 37 in the housing chamber R1 from the side of the exposed surface of the support wall 31b, the inspection work of inspecting the presence or absence, the state, and the like of the heat conduction member 37 can be simplified.

Furthermore, the support 31 may have the through hole R1a connected to the housing chamber R1 from the exposed surface of the support 31 (see FIG. 9 and FIG. 10). As a result, since the worker, the inspector, and the like of the manufacturing process can visually confirm the heat conduction member 37 in the housing chamber R1 from the side of the exposed surface of the support wall 31b, the inspection work of inspecting the presence or absence, and the like of the heat conduction member 37 can be simplified.

In addition, the excitation light source 12 may further include the first heat insulating member 41 provided between the support 31 and the substrate 35 in such a manner as to surround the temperature detecting section 36 and the heat conduction member 37 (see FIG. 11). As a result, since it becomes possible to control the dew condensation on the temperature detecting section 36 and the substrate 35 due to the heat conduction of the support 31 and to control erroneous detection of the temperature by the temperature detecting section 36 due to the dew condensation, the temperature detecting section 36 can accurately detect the temperature.

In addition, the excitation light source 12 may further include the optical member 43 provided on the surface of the light emitting section 32 which surface is on the side opposite to the support 31, and the second heat insulating member 42 provided between the support 31 and the optical member 43 in such a manner as to surround the light emitting section 32 (see FIG. 11). As a result, the dew condensation on the optical member 43 due to the heat conduction of the support 31 can be controlled.

In addition, the excitation light source 12 may further include the optical member 43 provided on the surface of the light emitting section 32 which surface is on the side opposite to the support 31, the first heat insulating member 41 provided between the support 31 and the substrate 35 in such a manner as to surround the temperature detecting section 36 and the heat conduction member 37, and the second heat insulating member 42 provided between the support 31 and the optical member 43 in such a manner as to surround the light emitting section 32 (see FIG. 11). As a result, since it becomes possible to control the dew condensation on the temperature detecting section 36 and the substrate 35 due to the heat conduction of the support 31 and to control erroneous detection of the temperature by the temperature detecting section 36 due to the dew condensation, the temperature detecting section 36 can accurately detect the temperature. Furthermore, the dew condensation on the optical member 43 due to the heat conduction of the support 31 can be controlled.

In addition, the first heat insulating member 41 may have the housing chamber R1 that houses the temperature detecting section 36 (see FIG. 11 and FIG. 12). As a result, even when the temperature detecting section 36 is provided at any position on the surface on the side of the support 31 of the substrate 35, the housing chamber R1 may be formed in accordance with the position, and a degree of freedom in installation of the temperature detecting section 36 can be improved.

In addition, the housing chamber R1 may be the cutout part formed in the first heat insulating member 41 (see FIG. 11 and FIG. 12). Thus, the housing chamber R1 can be easily formed.

The cutout part may be formed by cutting out of the exposed surface of the first heat insulating member 41 (see FIG. 11 and FIG. 12). As a result, since the worker, the inspector, and the like of the manufacturing process can visually confirm the heat conduction member 37 in the housing chamber R1 from the side of the exposed surface of the first heat insulating member 41, the inspection work of inspecting the presence or absence, the state, and the like of the heat conduction member 37 can be simplified.

Furthermore, the temperature detecting section 36 and the light emitting section 32 may be electrically connected to the substrate 35 (see FIG. 3). As a result, the electrical connection between the temperature detecting section 36 and the light emitting section 32 can be simplified.

In addition, the excitation light source 12 may further include the temperature control section 33 that is provided in the support 31 and that controls the temperature of the support 31 (see FIG. 3). As a result, since the temperature control section 33 is integrated with the support 31 and the excitation light source 12 is packaged, it is possible to improve a degree of freedom in installation of the excitation light source 12 and to simplify the installation.

Furthermore, the excitation light source 12 may further include a control section that is provided on the substrate 35 and that controls the temperature control section 33 on the basis of the temperature detected by the temperature detecting section 36 (such as the excitation light source control section 15) (see FIG. 1 and FIG. 3). As a result, since the control section is integrated with the substrate 35 and the excitation light source 12 is packaged, it is possible to improve the degree of freedom in installation of the excitation light source 12 and simplify the installation.

In addition, the excitation light source 12 may further include the heat dissipation section 34 that is provided in the temperature control section 33 and that dissipates heat (see FIG. 3). Since this makes it possible to dissipate the heat of the temperature control section 33 at the time of cooling, stable temperature control can be realized.

Furthermore, the heat dissipation section 34 may be provided with the temperature control section 33 being interposed with respect to the support 31 (see FIG. 3). As a result, the heat of the temperature control section 33 at the time of cooling can be securely dissipated. Thus, more stable temperature control can be realized.

In addition, the support 31 and the heat dissipation section 34 may be fixed by the plurality of fixing members 61 to 64, and the excitation light source 12 may further include the plurality of heat insulating members 71 to 74 that is provided between the support 31 and the heat dissipation section 34, prevents contact between the plurality of fixing members 61 to 64 and the heat dissipation section 34, and determines the separation distance between the support 31 and the heat dissipation section 34 (see FIG. 14 and FIG. 15). As a result, since the heat insulating members 71 to 74 respectively control the thermal collision in the fixing members 61 to 64, the efficiency of the temperature control section 33 can be improved. In addition, since each of the heat insulating members 71 to 74 makes the vertical separation distance between the support 31 and the heat dissipation section 34 constant, the height of the emission light (such as laser light) of the light emitting section 32 can be fixed.

In addition, the support 31 and the heat dissipation section 34 may be fixed by the plurality of fixing members 61A to 64A and 61B to 64B, and the excitation light source 12 may further include the plurality of heat insulating members 71A and 71B that is provided across the support 31 and the heat dissipation section 34, prevents the contact between the plurality of fixing members 61A to 64A and 61B to 64B, and determines the separation distance between the support 31 and the heat dissipation section 34 (FIG. 16 and FIG. 17). As a result, since the heat insulating members 71A and 71B respectively control the thermal collision in the fixing members 61A to 64A and 61B to 64B, the efficiency of the temperature control section 33 can be improved. In addition, since each of the heat insulating members 71A and 71B make the vertical separation distance between the support 31 and the heat dissipation section 34 constant, the height of the emission light (such as laser light) of the light emitting section 32 can be fixed.

Furthermore, a plurality of the light emitting sections 32 may be provided (for example, the light emitting sections 32A, 32B, and 32C may be provided), and the temperature detecting section 36 may be provided between the plurality of light emitting sections 32 (for example, between the light emitting sections 32B and 32C) (see FIG. 18). As a result, the temperature detecting section 36 can be disposed at a position where the temperature easily rises in the support 31, for example, between the two light emitting sections 32B and 32C. Thus, even when the temperature detecting section 36 is not installed for each of the light emitting sections 32, each of the light emitting sections 32 (such as each of the light emitting sections 32A, 32B, and 32C) can be used at the desired temperature (for example, within the desired temperature range) by the temperature control based on the one temperature detecting section 36.

### 9. Other Embodiments

A configuration according to each of the above-described embodiments (or modification examples) may be implemented in various different modes (modification examples) other than each of the above-described embodiments. In addition, the above-described embodiments (or modification examples) can be appropriately combined within a range in which the configuration contents do not contradict. Also, the effects described in the present description are merely examples and are not limitations, and there may be another effect.

### 10. Application Example

The technology according to the present disclosure can be applied to a medical imaging system. The medical imaging system is a medical system using an imaging technology, and is, for example, an endoscope system or a microscope system. The medical observation system 1 according to each of the above-described embodiments can be applied to the endoscope system or the microscope system. For example, the illumination device 10 of the medical observation system 1 can be applied to a light source device 5043, and the imaging device 20 of the medical observation system 1 can be applied to an endoscope 5001 and a microscope device 5301. Note that although basic operations, processing, and configurations will be described below with respect to the endoscope system and the microscope system, actually, the operations, processing, configurations, and the like according to the above-described embodiments are included.

### [Endoscope System]

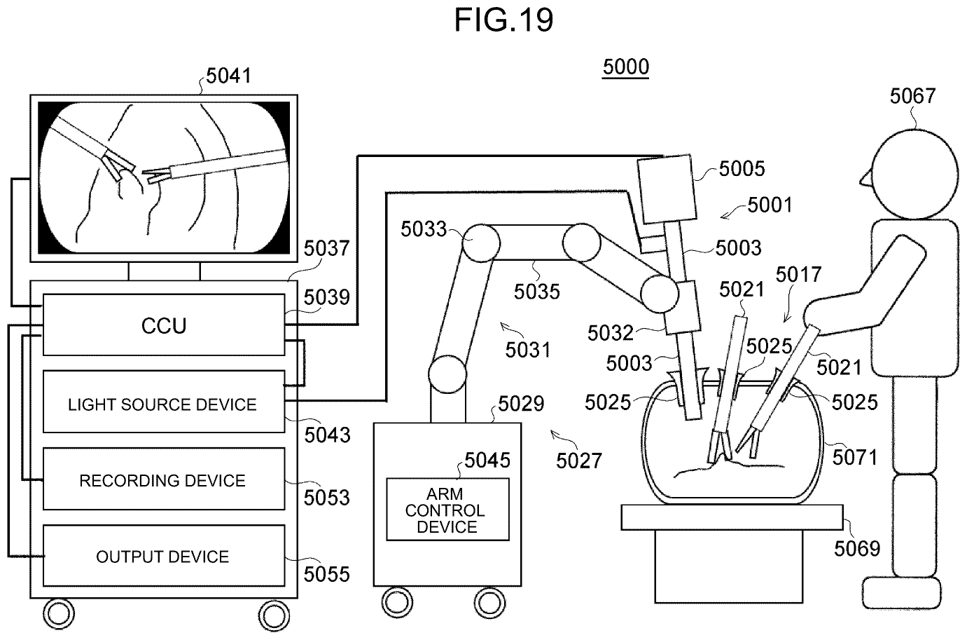

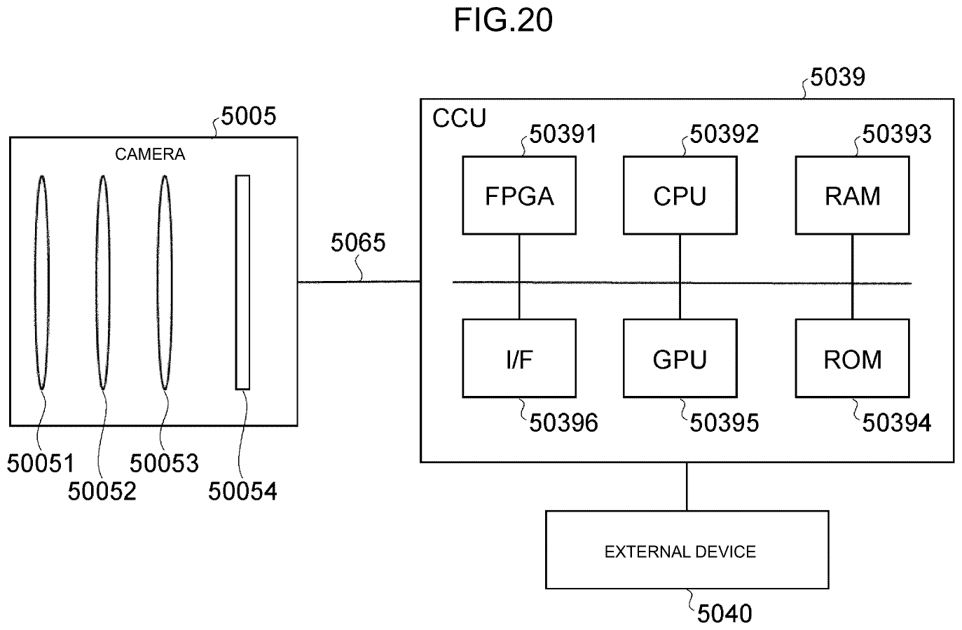

An example of the endoscope system will be described using FIGS. 19 and 20. FIG. 19 is a diagram illustrating an example of a schematic configuration of an endoscope system 5000 to which the technology according to the present disclosure is applicable. FIG. 20 is a diagram illustrating an example of a configuration of an endoscope 5001 and a camera control unit (CCU) 5039. FIG. 19 illustrates a situation where an operator (for example, a doctor) 5067 who is a participant of an operation performs the operation on a patient 5071 on a patient bed 5069 using the endoscope system 5000. As illustrated in FIG. 19, the endoscope system 5000 includes the endoscope 5001 that is a medical imaging device, the CCU 5039, a light source device 5043, a recording device 5053, an output device 5055, and a support device 5027 for supporting the endoscope 5001.

In endoscopic surgery, insertion assisting tools called trocars 5025 are punctured into the patient 5071. Then, a scope 5003 connected to the endoscope 5001 and surgical tools 5021 are inserted into a body of the patient 5071 through the trocars 5025. The surgical tools 5021 include: an energy device such as an electric scalpel; and forceps, for example.

A surgical image that is a medical image in which the inside of the body of the patient 5071 is captured by the endoscope 5001 is displayed on a display device 5041. The operator 5067 performs a procedure on a surgical target using the surgical tools 5021 while viewing the surgical image displayed on the display device 5041. The medical image is not limited to the surgical image, and may be a diagnostic image captured during diagnosis.

### [Endoscope]

The endoscope 5001 is an imaging section for capturing the inside of the body of the patient 5071, and is, for example, as illustrated in FIG. 20, a camera 5005 including a condensing optical system 50051 for condensing incident light, a zooming optical system 50052 capable of optical zooming by changing a focal length of the imaging section, a focusing optical system 50053 capable of focus adjustment by changing the focal length of the imaging section, and a light receiving sensor 50054. The endoscope 5001 condenses the light through the connected scope 5003 on the light receiving sensor 50054 to generate a pixel signal, and outputs the pixel signal through a transmission system to the CCU 5039. The scope 5003 is an insertion part that includes an objective lens at a distal end and guides the light from the connected light source device 5043 into the body of the patient 5071. The scope 5003 is, for example, a rigid scope for a rigid endoscope and a flexible scope for a flexible endoscope. The scope 5003 may be a direct viewing scope or an oblique viewing scope. The pixel signal only needs to be a signal based on a signal output from a pixel, and is, for example, a raw signal or an image signal. The transmission system connecting the endoscope 5001 to the CCU 5039 may include a memory, and the memory may store parameters related to the endoscope 5001 and the CCU 5039. The memory may be disposed at a connection portion of the transmission system or on a cable.

For example, the memory of the transmission system may store the parameters before shipment of the endoscope 5001 or the parameters changed when current is applied, and an operation of the endoscope may be changed based on the parameters read from the memory. A set of the camera and the transmission system may be referred to as an endoscope. The light receiving sensor 50054 is a sensor for converting the received light into the pixel signal, and is, for example, a complementary metal-oxide-semiconductor (CMOS) imaging sensor. The light receiving sensor 50054 is preferably an imaging sensor having a Bayer array capable of color imaging. The light receiving sensor 50054 is also preferably an imaging sensor having a number of pixels corresponding to a resolution of, for example, 4K (3840 horizontal pixels×2160 vertical pixels), 8K (7680 horizontal pixels×4320 vertical pixels), or square 4K (3840 or more horizontal pixels×3840 or more vertical pixels). The light receiving sensor 50054 may be one sensor chip, or a plurality of sensor chips. For example, a prism may be provided to separate the incident light into predetermined wavelength bands, and the wavelength bands may be imaged by different light receiving sensors. A plurality of light receiving sensors may be provided for stereoscopic viewing. The light receiving sensor 50054 may be a sensor having a chip structure including an arithmetic processing circuit for image processing, or may be a sensor for time of flight (ToF). The transmission system is, for example, an optical fiber cable system or a wireless transmission system. The wireless transmission only needs to be capable of transmitting the pixel signal generated by the endoscope 5001, and, for example, the endoscope 5001 may be wirelessly connected to the CCU 5039, or the endoscope 5001 may be connected to the CCU 5039 via a base station in an operating room. At this time, the endoscope 5001 may transmit not only the pixel signal, but also simultaneously information (for example, a processing priority of the pixel signal and/or a synchronization signal) related to the pixel signal. In the endoscope, the scope may be integrated with the camera, and the light receiving sensor may be provided at the distal end of the scope.

### [Camera Control Unit (CCU)]

The CCU 5039 is a control device for controlling the endoscope 5001 and the light source device 5043 connected to the CCU 5039 in an integrated manner, and is, for example, as illustrated in FIG. 20, an image processing device including a field-programmable gate array (FPGA) 50391, a central processing unit (CPU) 50392, a random access memory 50393, a read-only memory (ROM) 50394, a graphics processing unit (GPU) 50395, and an interface (I/F) 50396. The CCU 5039 may control the display device 5041, the recording device 5053, and the output device 5055 connected to the CCU 5039 in an integrated manner. The CCU 5039 controls, for example, irradiation timing, irradiation intensity, and a type of an irradiation light source of the light source device 5043. The CCU 5039 also performs image processing, such as development processing (for example, demosaic processing) and correction processing, on the pixel signal output from the endoscope 5001, and outputs the processed image signal (for example, an image) to an external device such as the display device 5041. The CCU 5039 also transmits a control signal to the endoscope 5001 to control driving of the endoscope 5001. The control signal is information on an imaging condition such as a magnification or the focal length of the imaging section. The CCU 5039 may have a function to down-convert the image, and may be configured to be capable of simultaneously outputting a higher-resolution (for example, 4K) image to the display device 5041 and a lower-resolution (for example, high-definition (HD)) image to the recording device 5053.

The CCU 5039 may be connected to external equipment (such as a recording device, a display device, an output device, and a support device) via an IP converter for converting the signal into a predetermined communication protocol (such as the Internet Protocol (IP)). The connection between the IP converter and the external equipment may be established using a wired network, or a part or the whole of the network may be established using a wireless network. For example, the IP converter on the CCU 5039 side may have a wireless communication function, and may transmit the received image to an IP switcher or an output side IP converter via a wireless communication network, such as the fifth-generation mobile communication system (5G) or the sixth-generation mobile communication system (6G).

### [Light Source Device]

The light source device 5043 is a device capable of emitting the light having predetermined wavelength bands, and includes, for example, a plurality of light sources and a light source optical system for guiding the light of the light sources. The light sources are, for example, xenon lamps, light-emitting diode (LED) light sources, or laser diode (LD) light sources. The light source device 5043 includes, for example, the LED light sources corresponding to three respective primary colors of red (R), green (G), and blue (B), and controls output intensity and output timing of each of the light sources to emit white light. The light source device 5043 may include a light source capable of emitting special light used for special light observation, in addition to the light sources for emitting normal light for normal light observation. The special light is light having a predetermined wavelength band different from that of the normal light being light for the normal light observation, and is, for example, near-infrared light (light having a wavelength of 760 nm or longer), infrared light, blue light, or ultraviolet light. The normal light is, for example, the white light or green light. In narrow band imaging that is a kind of special light observation, blue light and green light are alternately emitted, and thus the narrow band imaging can image a predetermined tissue such as a blood vessel in a mucosal surface at high contrast using wavelength dependence of light absorption in the tissue of the body. In fluorescence observation that is a kind of special light observation, excitation light is emitted for exciting an agent injected into the tissue of the body, and fluorescence emitted by the tissue of the body or the agent as a label is received to obtain a fluorescent image, and thus the fluorescence observation can facilitate the operator to view, for example, the tissue of the body that is difficult to be viewed by the operator with the normal light. For example, in fluorescence observation using the infrared light, the infrared light having an excitation wavelength band is emitted to an agent, such as indocyanine green (ICG), injected into the tissue of the body, and the fluorescence light from the agent is received, whereby the fluorescence observation can facilitate viewing of a structure and an affected part of the tissue of the body. In the fluorescence observation, an agent (such as 5-aminolevulinic acid (5-ALA)) may be used that emits fluorescence in a red wavelength band by being excited by the special light in a blue wavelength band. The type of the irradiation light of the light source device 5043 is set by control of the CCU 5039. The CCU 5039 may have a mode of controlling the light source device 5043 and the endoscope 5001 to alternately perform the normal light observation and the special light observation. At this time, information based on a pixel signal obtained by the special light observation is preferably superimposed on a pixel signal obtained by the normal light observation. The special light observation may be an infrared light observation to observe a site inside the surface of an organ and a multi-spectrum observation utilizing hyperspectral spectroscopy. A photodynamic therapy may be incorporated.

### [Recording Device]

The recording device 5053 is a device for recording the pixel signal (for example, an image) acquired from the CCU 5039, and is, for example, a recorder. The recording device 5053 records an image acquired from the CCU 5039 in a hard disk drive (HDD), a Super Density Disc (SDD), and/or an optical disc. The recording device 5053 may be connected to a network in a hospital to be accessible from equipment outside the operating room. The recording device 5053 may have a down-convert function or an up-convert function.

### [Display Device]

The display device 5041 is a device capable of displaying the image, and is, for example, a display monitor. The display device 5041 displays a display image based on the pixel signal acquired from the CCU 5039. The display device 5041 may include a camera and a microphone to function as an input device that allows instruction input through gaze recognition, voice recognition, and gesture.

### [Output Device]

The output device 5055 is a device for outputting the information acquired from the CCU 5039, and is, for example, a printer. The output device 5055 prints, for example, a print image based on the pixel signal acquired from the CCU 5039 on a sheet of paper.

### [Support Device]

The support device 5027 is an articulated arm including a base 5029 including an arm control device 5045, an arm 5031 extending from the base 5029, and a holding part 5032 mounted at a distal end of the arm 5031. The arm control device 5045 includes a processor such as a CPU, and operates according to a predetermined computer program to control driving of the arm 5031. The support device 5027 uses the arm control device 5045 to control parameters including, for example, lengths of links 5035 constituting the arm 5031 and rotation angles and torque of joints 5033 so as to control, for example, the position and attitude of the endoscope 5001 held by the holding part 5032. This control can change the position or attitude of the endoscope 5001 to a desired position or attitude, makes it possible to insert the scope 5003 into the patient 5071, and can change the observed area in the body. The support device 5027 functions as an endoscope support arm for supporting the endoscope 5001 during the operation. Thus, the support device 5027 can play a role of a scopist who is an assistant holding the endoscope 5001. The support device 5027 may be a device for holding a microscope device 5301 to be described later, and can be called a medical support arm. The support device 5027 may be controlled using an autonomous control method by the arm control device 5045, or may be controlled using a control method in which the arm control device 5045 performs the control based on input of a user. The control method may be, for example, a master-slave method in which the support device 5027 serving as a slave device (replica device) that is a patient cart is controlled based on a movement of a master device (primary device) that is an operator console at a hand of the user. The support device 5027 may be remotely controllable from outside the operating room.

The example of the endoscope system 5000 to which the technology according to the present disclosure is applicable has been described above. For example, the technology according to the present disclosure may be applied to a microscope system.

### [Microscope System]

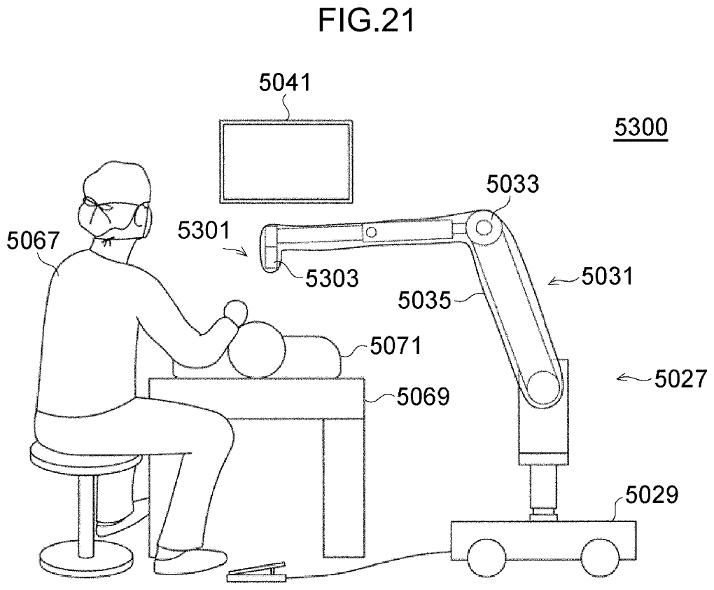

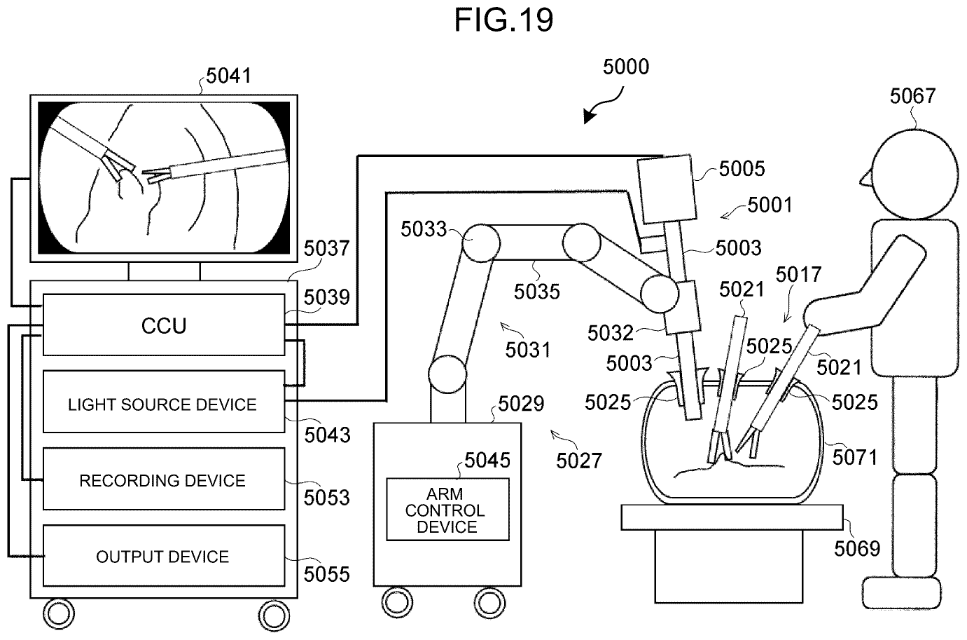

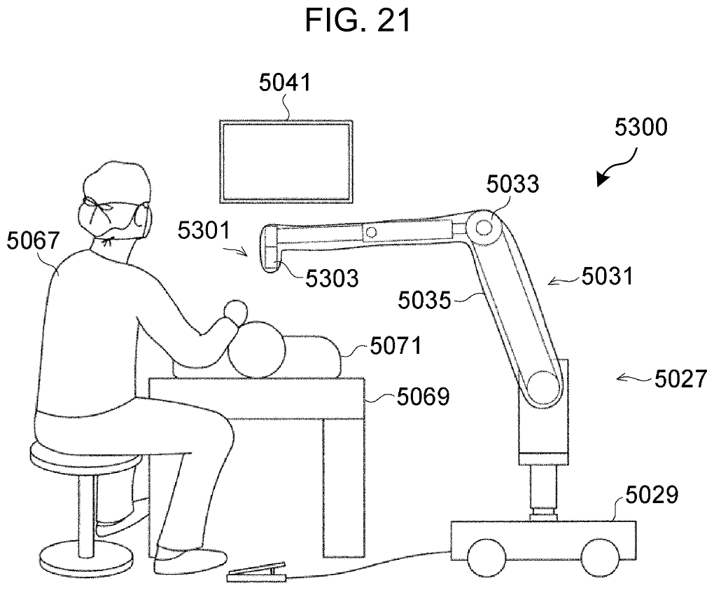

FIG. 21 is a diagram illustrating an example of a schematic configuration of a microscopic surgery system to which the technology according to the present disclosure is applicable. In the following description, the same components as those of the endoscope system 5000 will be denoted by the same reference numerals, and the description thereof will not be repeated.

FIG. 21 schematically illustrates a situation where the operator 5067 performs an operation on the patient 5071 on the patient bed 5069 using a microscopic surgery system 5300. For the sake of simplicity, FIG. 21 does not illustrate a cart 5037 among the components of the microscopic surgery system 5300, and illustrates the microscope device 5301 instead of the endoscope 5001 in a simplified manner. The microscope device 5301 may refer to a microscope 5303 provided at the distal end of the links 5035, or may refer to the overall configuration including the microscope 5303 and the support device 5027.

As illustrated in FIG. 21, during the operation, the microscopic surgery system 5300 is used to display an image of a surgical site captured by the microscope device 5301 in a magnified manner on the display device 5041 installed in the operating room. The display device 5041 is installed in a position facing the operator 5067, and the operator 5067 performs various procedures, such as excision of an affected part, on the surgical site while observing the state of the surgical site using the image displayed on the display device 5041. The microscopic surgery system is used in, for example, ophthalmic operation and neurosurgical operation.

The respective examples of the endoscope system 5000 and the microscopic surgery system 5300 to which the technology according to the present disclosure is applicable have been described above. Systems to which the technology according to the present disclosure is applicable are not limited to such examples. For example, the support device 5027 can support, at the distal end thereof, another observation device or another surgical tool instead of the endoscope 5001 or the microscope 5303. Examples of the other applicable observation device include forceps, tweezers, a pneumoperitoneum tube for pneumoperitoneum, and an energy treatment tool for incising a tissue or sealing a blood vessel by cauterization. By using the support device to support the observation device or the surgical tool described above, the position thereof can be more stably fixed and the load of the medical staff can be lower than in a case where the medical staff manually supports the observation device or the surgical tool. The technology according to the present disclosure may be applied to a support device for supporting such a component other than the microscope.

The technology according to the present disclosure can be suitably applied to the endoscope 5001, the microscope device 5301, the CCU 5039, the light source device 5043, and the like among the above-described configurations. Specifically, the operation and processing according to each of the embodiments can be executed in the endoscope system 5000, the microscopic surgery system 5300, and the like. By applying the technology according to the present disclosure to the endoscope system 5000, the microscopic surgery system 5300, and the like, stable temperature control and improvement in mass productivity can be realized.

### 11. Supplementary Note

Note that the present technology can also have the following configurations.

(1)

A light source device comprising:
a support;
a substrate provided on the support;
a temperature detecting section that is provided on a surface on a side of the support of the substrate and detects temperature;
a light emitting section that is provided on a surface of the support, the surface being on a side opposite to the substrate, and that emits light; and
a heat conduction member provided between the temperature detecting section and the support in such a manner as to be in contact with the temperature detecting section and the support.

(2)

The light source device according to (1), wherein
the support includes a housing chamber that houses the temperature detecting section.

(3)

The light source device according to (2), wherein
the housing chamber is a recess part formed in a surface on a side of the substrate of the support.

(4)

The light source device according to (3), wherein
the recess part is formed in such a manner as to extend to an exposed surface of the support.

(5)

The light source device according to (2), wherein
the support has a through hole connected to the housing chamber from an exposed surface of the support.

(6)

The light source device according to any one of (1) to (5), further comprising
a first heat insulating member provided between the support and the substrate in such a manner as to surround the temperature detecting section and the heat conduction member.

(7)

The light source device according to any one of (1) to (5), further comprising:
an optical member provided on a surface of the light emitting section which surface is opposite to the side of the support; and
a second heat insulating member provided between the support and the optical member in such a manner as to surround the light emitting section.

(8)

The light source device according to any one of (1) to (5), further comprising:

an optical member provided on a surface of the light emitting section which surface is opposite to the side of the support;
a first heat insulating member provided between the support and the substrate in such a manner as to surround the temperature detecting section and the heat conduction member; and
a second heat insulating member provided between the support and the optical member in such a manner as to surround the light emitting section.

(9)

The light source device according to any one of (6) to (8), wherein
the first heat insulating member includes a housing chamber that houses the temperature detecting section.

(10)

The light source device according to (9), wherein
the housing chamber is a cutout part formed in the first heat insulating member.

(11)

The light source device according to (10), wherein
the cutout part is formed by cutting out of an exposed surface of the first heat insulating member.

(12)

The light source device according to any one of (1) to (11), wherein
the temperature detecting section and the light emitting section are electrically connected to the substrate.

(13)

The light source device according to any one of (1) to (12), further comprising
a temperature control section that is provided on the support and adjusts a temperature of the support.

(14) The light source device according to (13), further comprising
a control section that is provided on the substrate and controls the temperature control section on a basis of the temperature detected by the temperature detecting section.

(15)

The light source device according to (13) or (14), further comprising
a heat dissipation section that is provided in the temperature control section and dissipates heat.

(16)

The light source device according to (15), wherein
the heat dissipation section is provided with the temperature control section being interposed with respect to the support.

(17)

The light source device according to (16), wherein
the support and the heat dissipation section are fixed by a plurality of fixing members, and
the light source device further comprises a plurality of heat insulating members that is provided between the support and the heat dissipation section, prevents contact between the plurality of fixing members and the heat dissipation section, and determines a separation distance between the support and the heat dissipation section.

(18)

The light source device according to (16), wherein
the support and the heat dissipation section are fixed by a plurality of fixing members, and
the light source device further comprises a plurality of heat insulating members that is provided across the support and the heat dissipation section, prevents contact between the plurality of fixing members, and determines a separation distance between the support and the heat dissipation section.

(19)

The light source device according to any one of (1) to (18), wherein
a plurality of the light emitting sections is provided, and
the temperature detecting section is provided between the plurality of light emitting sections.

(20)

A medical observation system comprising:
an imaging device that images an imaging target; and
a light source device that generates light emitted to the imaging target, wherein
the light source device includes
a support,
a substrate provided on the support,
a temperature detecting section that is provided on a surface on a side of the support of the substrate and that detects temperature,
a light emitting section that is provided on a surface of the support, the surface being opposite to a side of the substrate, and that emits light, and
a heat conduction member provided between the temperature detecting section and the support in such a manner as to be in contact with the temperature detecting section and the support.

(21)

A medical observation system including the light source device according to any one of (1) to (19).

(22)

A medical observation method using the light source device according to any one of (1) to (19).

### REFERENCE SIGNS LIST

1 MEDICAL OBSERVATION SYSTEM
2 IMAGING TARGET
10 ILLUMINATION DEVICE
11 WHITE LIGHT SOURCE
12 EXCITATION LIGHT SOURCE
12a EXCITATION LIGHT SOURCE
12b EXCITATION LIGHT SOURCE
13 MULTIPLEXING SYSTEM
14 WHITE LIGHT SOURCE CONTROL SECTION
15 EXCITATION LIGHT SOURCE CONTROL SECTION
16 LIGHT SOURCE CONTROL SECTION
20 IMAGING DEVICE
21 OPTICAL SYSTEM
22 LIGHT RECEIVING SECTION
23 IMAGING PROCESSING SECTION
31 SUPPORT
31a SUPPORT PLATE
31b SUPPORT WALL
32 LIGHT EMITTING SECTION
32A LIGHT EMITTING SECTION
32B LIGHT EMITTING SECTION
32C LIGHT EMITTING SECTION
32a LIGHT EMITTING ELEMENT
32b COVER PART
32c TERMINAL
32d TERMINAL
32e TERMINAL
33 TEMPERATURE CONTROL SECTION
34 HEAT DISSIPATION SECTION
35 SUBSTRATE
35a THROUGH HOLE
35b THROUGH HOLE
35c THROUGH HOLE
36 TEMPERATURE DETECTING SECTION
37 HEAT CONDUCTION MEMBER
41 FIRST HEAT INSULATING MEMBER
41a THROUGH HOLE
41b THROUGH HOLE
41c THROUGH HOLE
42 SECOND HEAT INSULATING MEMBER
43 OPTICAL MEMBER
43a LENS
51 GREASE LAYER
52 GREASE LAYER
61 FIXING MEMBER
61A FIXING MEMBER
61B FIXING MEMBER
62 FIXING MEMBER
62A FIXING MEMBER
62B FIXING MEMBER
63 FIXING MEMBER
63A FIXING MEMBER
63B FIXING MEMBER
64 FIXING MEMBER
64A FIXING MEMBER
64B FIXING MEMBER
71 HEAT INSULATING MEMBER
71A HEAT INSULATING MEMBER
71B HEAT INSULATING MEMBER
72 HEAT INSULATING MEMBER
73 HEAT INSULATING MEMBER
74 HEAT INSULATING MEMBER
A1 HEAT CONDUCTION PATH
A2 HEAT CONDUCTION PATH
R1 HOUSING CHAMBER
R1a THROUGH HOLE

What is claimed is:

1. A light source device comprising:
a support;
a substrate provided on the support;
a temperature detecting section that is provided on a surface on a side of the support of the substrate and detects temperature;
a light emitting section that is provided on a surface of the support, the surface being on a side opposite to the substrate, and that emits light; and
a heat conduction member provided between the temperature detecting section and the support in such a manner as to be in contact with the temperature detecting section and the support.

2. The light source device according to claim 1, wherein
the support includes a housing chamber that houses the temperature detecting section.

3. The light source device according to claim 2, wherein
the housing chamber is a recess part formed in a surface on a side of the substrate of the support.

4. The light source device according to claim 3, wherein
the recess part is formed in such a manner as to extend to an exposed surface of the support.

5. The light source device according to claim 2, wherein
the support has a through hole connected to the housing chamber from an exposed surface of the support.

6. The light source device according to claim 1, further comprising
a first heat insulating member provided between the support and the substrate in such a manner as to surround the temperature detecting section and the heat conduction member.

7. The light source device according to claim 1, further comprising:
an optical member provided on a surface of the light emitting section which surface is opposite to the side of the support; and
a second heat insulating member provided between the support and the optical member in such a manner as to surround the light emitting section.

8. The light source device according to claim 1, further comprising:
an optical member provided on a surface of the light emitting section which surface is opposite to the side of the support;
a first heat insulating member provided between the support and the substrate in such a manner as to surround the temperature detecting section and the heat conduction member; and
a second heat insulating member provided between the support and the optical member in such a manner as to surround the light emitting section.

9. The light source device according to claim 6, wherein
the first heat insulating member includes a housing chamber that houses the temperature detecting section.

10. The light source device according to claim 9, wherein
the housing chamber is a cutout part formed in the first heat insulating member.

11. The light source device according to claim 10, wherein
the cutout part is formed by cutting out of an exposed surface of the first heat insulating member.

12. The light source device according to claim 1, wherein
the temperature detecting section and the light emitting section are electrically connected to the substrate.

13. The light source device according to claim 1, further comprising
a temperature control section that is provided on the support and adjusts a temperature of the support.

14. The light source device according to claim 13, further comprising
a control section that is provided on the substrate and controls the temperature control section on a basis of the temperature detected by the temperature detecting section.

15. The light source device according to claim 13, further comprising
a heat dissipation section that is provided in the temperature control section and dissipates heat.

16. The light source device according to claim 15, wherein
the heat dissipation section is provided with the temperature control section being interposed with respect to the support.

17. The light source device according to claim 16, wherein
the support and the heat dissipation section are fixed by a plurality of fixing members, and
the light source device further comprises a plurality of heat insulating members that is provided between the support and the heat dissipation section, prevents contact between the plurality of fixing members and the heat dissipation section, and determines a separation distance between the support and the heat dissipation section.

18. The light source device according to claim 16, wherein
the support and the heat dissipation section are fixed by a plurality of fixing members, and
the light source device further comprises a plurality of heat insulating members that is provided across the support and the heat dissipation section, prevents contact between the plurality of fixing members, and determines a separation distance between the support and the heat dissipation section.

19. The light source device according to claim 1, wherein
a plurality of the light emitting sections is provided, and
the temperature detecting section is provided between the plurality of light emitting sections.

20. A medical observation system comprising:
an imaging device that images an imaging target; and
a light source device that generates light emitted to the imaging target, wherein
the light source device includes
a support,
a substrate provided on the support,
a temperature detecting section that is provided on a surface on a side of the support of the substrate and that detects temperature,
a light emitting section that is provided on a surface of the support, the surface being opposite to a side of the substrate, and that emits light, and
a heat conduction member provided between the temperature detecting section and the support in such a manner as to be in contact with the temperature detecting section and the support.
